(19) United States
(12) Patent Application Publication
(10) Pub. No.: US 2005/0276777 A1
(43) Pub. Date: Dec. 15, 2005

(54) Detergent composition comprising at least one nonionic surfactant and at least one anionic polymer and process for color protection therewith

(76) Inventors: Boris Lalleman, Paris (FR); Franck Giroud, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/118,952

(22) Filed: May 2, 2005

Related U.S. Application Data
(60) Provisional application No. 60/569,647, filed on May 11, 2004.

(30) Foreign Application Priority Data
Apr. 30, 2004 (FR) 04 04650

(51) Int. Cl. A61K 7/06; A61K 7/11

(52) U.S. Cl. 424/70.13; 424/70.16

(57) ABSTRACT

The present disclosure relates to washing compositions comprising at least one crosslinked anionic polymer and at least one nonionic surfactant present in an amount ranging from 5% to 20% by weight. It also relates to a process for washing keratin fibers with these compositions, and the use of these washing compositions for protecting the coloration of keratin fibers.

This application claims benefit of U.S. Provisional Application No. 60/569,647, filed May 11, 2004, and French Patent Application No. 04 04650, filed Apr. 30, 2004, the contents of both of which are herein incorporated by reference.

The present disclosure relates to a composition comprising at least one anionic polymer and at least one nonionic surfactant, and/or mixtures thereof, to a process for treating keratin fibers, such as dyed keratin fibers, and also to a method for protecting the coloration of said fibers.

In addition, the present disclosure also relates to the treatment of keratin fibers, for instance dyed keratin fibers, for example human keratin fibers, such as the hair.

For cleansing and/or washing the hair, the use of detergent compositions (or shampoos) based essentially on conventional surfactants of, for example, the anionic type, is known. These compositions can be applied to wet hair and the foam generated by massage or friction with the hands may make it possible, after rinsing with water, to remove the various forms of dirt initially present on the hair.

These base compositions admittedly can possess good washing power, but the intrinsic cosmetic properties attached to them can be, however, quite poor, for example due to the fact that the relatively aggressive nature of such cleansing treatment can, in the long term, result in more or less marked damage on the hair fiber, linked for instance, to the gradual removal of the lipids or proteins contained in or at the surface of the hair fiber.

In addition, on dyed hair, shampoos can bring about a phenomenon of bleeding of the color that may not be insignificant.

Surfactants of the family of alkylpolyglycosides or polyglycerol surfactants have been recommended for use in washing compositions for the hair or the skin. They can be mild, well-tolerated and biodegradable detergents.

Generally, the washing compositions can be thickened. Washing products that can be dispensed slowly and readily taken in the hand are sometimes sought by the consumer. To have these qualities, the washing products may have a certain consistency or viscosity. In practice, a liquid product can be more difficult to dispense at a controlled rate and can flow readily between the fingers, which may hamper the application to the hair. Moreover, in the case of shampoo, the product may flow onto the face and for example, into the eyes, which is not desirable.

However, the thickening of nonionic surfactant-based compositions can be difficult to carry out by conventional methods (such as coconut amides, electrolytes, etc.) due to their nonionic structure. Synthetic or natural polymers, such as crosslinked acrylic polymers of the Carbopol type, celluloses, etc. have already been used to thicken cosmetic compositions containing these surfactants. Unfortunately, most thickeners can give unstable and/or non-transparent compositions. In addition, certain thickeners, for instance associative thickeners containing fatty chains, can have the drawback of decreasing the quality of the foam and the cosmetic performance levels of these shampoos, for example, they may make the hair more charged and more harsh. The foam of the thickened compositions may not be sufficiently mild, and it may not develop easily, whether in terms of speed or of abundance.

Thus, there remains a need for a thickening system for suitably thickening a washing composition comprising at least one nonionic surfactant without impairing the cosmetic and foaming properties of the composition.

Accordingly, one aspect of the present disclosure is to obtain detergent compositions that may not degrade the color (coloration) of the hair, that may have good viscosity, may give an abundant fine foam, and can develop readily.

The present inventors have discovered, surprisingly, that the combination, in washing compositions for keratin substances, of at least one specific acrylic copolymer with at least one nonionic surfactant, for instance of the alkylpolyglycoside and/or polyglycerol-based type, can make it possible to obtain compositions that are thickened, stable, homogeneous and clear. Moreover, these compositions can make it possible to obtain a very mild foam. The initiation of the foam can be rapid and the foam can be stable and can rinse off readily.

Further, the washing composition may allow good protection of the coloration of dyed keratin fibers, such as hair. It may, in fact, enable less bleeding, and induce less stripping, of the coloration with each shampooing. It has also been noted, consequently, that lesser bleeding of the dye(s) can be reflected by a reduced risk of staining of the skin and of fabrics.

Thus, one aspect of the present disclosure is a detergent cosmetic composition comprising, in an aqueous medium, for instance a physiologically acceptable, such as a cosmetically acceptable medium, at least one nonionic surfactant, present in an amount ranging from 5% to 20% by weight, chosen from:

glycerol-based fatty alcohols;

glycerol-based fatty amides;

oxyalkylenated plant oils;

alkylpolyglycosides; and

derivatives of N-alkylglucamine;

and at least one crosslinked anionic copolymer, present in an amount ranging from 0.01% to 10% by weight relative. to the total weight of the composition, of vinyl carboxylic acid (monomer A) and of a monomer having at least one α,β-ethylenic unsaturation (monomer B) chosen from the monomers of formulae:
CH₂═CXY i)
wherein X is a hydrogen atom, and Y is chosen from —COOR, —C₆H₄R′, —CN, —CONH₂, —NHCOCH₃, —CONHC(CH₃)₃, and —CON(CH₃)₂ groups; or

X is a CH₃ groups and Y is chosen from —COOR, —C₆H₄R′, —CN and —CH═CH₂ groups,

R is chosen from C₁-C₈ alkyl and C₂-C₈ hydroxyalkyl groups,

R′ is chosen from a hydrogen atom, and C₁-C₈ alkyl and C₂-C₈ hydroxyalkyl groups;
CH₂═CH(OCOR¹) ii)
wherein R¹ is chosen from C₁-C₈, such as C₁-C₄, alkyl groups; and
CH₂═CH₂ or CH₂═CHCH₃. iii)

Still another aspect of the present disclosure is the use of a composition as disclosed herein, for limiting the “bleeding” of the coloration and/or protecting the coloration of keratin fibers.

The present disclosure also relates to a process for treating dyed keratin fibers that can make it possible to reduce bleeding of the dyed color during shampooing, while at the same time benefiting from good qualities in terms of shampoo use.

Still yet another aspect of the present disclosure comprises the washing and/or conditioning processes using such compositions.

Other benefits and characteristics of the present disclosure will emerge more clearly upon reading the description and the examples, which follow.

The at least one copolymer as disclosed herein comprises at least one monomer A, at least one monomer B, and at least one crosslinking agent.

Non-limiting examples of the vinyl carboxylic acids (monomers A) that may be used include those chosen from acrylic acid, methacrylic acid, itaconic acid, fumaric acid, crotonic acid, maleic acid and mixtures thereof. For example, the vinyl carboxylic acids may be chosen from acrylic acid and methacrylic acid, such as methacrylic acid.

The monomers B having at least one α,β-ethylenic unsaturation can be chosen from the monomers of formulae:
CH₂═CXY i)
wherein X is a hydrogen atom and Y is chosen from —COOR, —C₆H₄R′, —CN, —CONH₂, —NHCOCH₃, —CONHC(CH₃)₃, and —CON(CH₃)₂ groups, or

X is a CH₃ group and Y is chosen from —COOR, —C₆H₄R′, —CN and —CH═CH₂ groups,

R is chosen from C₁-C₈ alkyl and C₂-C₈ hydroxyalkyl groups,

R′ is chosen from a hydrogen atom, and C₁-C₈ alkyl and C₂-C₈ hydroxyalkyl groups;
CH₂═CH(OCOR¹) ii)
wherein R¹ is chosen from C₁-C₈, such as C₁-C₄, alkyl groups; and
CH₂═CH₂ or CH₂═CHCH₃. iii)

For example, R may be chosen from C₁-C₄ alkyl and C₂-C₄ hydroxyalkyl groups.

The at least one monomer B having at least one α,β-ethylenic unsaturation may be, for example, a monomer of formula i). Among the monomers from which the at least one monomer B may be chosen, non-limiting mention may be made of methyl acrylate, ethyl acrylate, n-butyl acrylate, 2-ethylhexyl acrylate, 2-hydroxyethyl acrylate, styrene, vinyl acetates, acrylamide, N,N-dimethylacrylamide, tert-butylacrylamide, methyl methacrylate, ethyl methacrylate, n-butyl methacrylate, 2-ethylhexyl methacrylate and 2-hydroxyethyl methacrylate.

The at least one monomer B having at least one α,β-ethylenic unsaturation may also be, for instance, a monomer of formula i) wherein X is a hydrogen atom and Y is a —COOR group. For example, the monomer B may be chosen from methyl acrylate, ethyl acrylate and butyl acrylate groups, such as ethyl acrylate.

The at least one monomer A can be present, for example, in an amount ranging from 20% to 80% by weight, for instance, from 25% to 70% by weight, such as from 35% to 60% by weight, relative to the total weight of the copolymer.

The at least one monomer B is can be present, for example, in an amount ranging from 15% to 80% by weight, for instance from 25% to 75% by weight, such as from 40% to 65% by weight, relative to the total weight of the copolymer.

This copolymer is partially or completely crosslinked with at least one conventional crosslinking agent. Non-limiting examples of crosslinking agents include polyunsaturated compounds, such as ethylenically polyunsaturated compounds. Among these compounds, non-limiting mention may be made of poly(alkenyl ether)s of sucrose or of polyols, diallyl phthalates, divinylbenzene, allyl (meth)acrylate, ethylene glycol di(meth)acrylate, methylenebisacrylamide, trimethylolpropane tri(meth)acrylate, diallyl itaconate, diallyl fumarate, diallyl maleate, zinc (meth)acrylate, or derivatives of castor oil or of polyols produced from unsaturated carboxylic acids.

As a crosslinking agent, use may also be made of unsaturated monomeric compounds comprising a reactive group capable of reacting with an unsaturation so as to form a crosslinked copolymer.

The at least one crosslinking agent can be present in an amount ranging from 0.01% to 5% by weight, for instance from 0.03% to 3% by weight, such as from 0.05% to 1% by weight, relative to the total weight of the copolymer.

The copolymer according to the present disclosure may be chosen from, for example, a crosslinked copolymer of methacrylic acid and of C₁-C₄ alkyl acrylate, a crosslinked copolymer of acrylic acid and of C₁-C₄ alkyl acrylate, and for instance, a crosslinked copolymer of methacrylic acid and of ethyl acrylate.

According to one embodiment of the present disclosure, the copolymer as disclosed herein may be, for example, in the form of a dispersion in water. The number-average size of the particles of copolymer in the dispersion can range from 10 nm to 500 nm, such as from 20 nm to 200 nm, and for instance from 50 nm to 150 nm. These copolymers are described, for example, in International Patent Application No. WO 01/76552.

In another embodiment of the present disclosure, use can be made of a methacrylic acid/ethyl acrylate crosslinked copolymer in the form of a 30% aqueous dispersion, produced and sold under the name CARBOPOL AQUA SF-1 by the company Noveon, or else a crosslinked copolymer of (meth)acrylic acid and of C₁-C₄ alkyl acrylate sold under the name ACULYN 33 by the company Rohm & Haas.

The at least one crosslinked copolymer can be present in an amount ranging from 0.01% to 10% by weight, relative to the total weight of the composition, for instance from 0.1% to 5% by weight, such as from 0.5% to 3% by weight.

The detergent composition comprises, as surfactant, at least one nonionic surfactant.

For example, by way of nonionic surfactants, non-limiting mention may be made of:

glycerol-based fatty alcohols;

alkylpolyglycosides.

As used herein, the term “fatty chain” is understood to mean a linear or branched, saturated or unsaturated hydrocarbon-based chain comprising from 6 to 30 carbon atoms, such as from 8 to 24 carbon atoms.

With respect to the glycerol-based surfactants, they can be monoglycerol-based or polyglycerol-based, and they can comprise, for example, from 1 to 5 glycerol groups, such as from 1.5 to 4 glycerol groups.

The washing composition as disclosed herein can comprise at least one nonionic surfactant chosen from, for instance, alkylpolyglycosides, monoglycerol-based surfactants and polyglycerol-based surfactants.

With respect to the alkylpolyglycosides, these compounds are well known and can be chosen from those of formula (II):
R₁O—(R₂O)ₜ(G)ᵥ (II)
wherein

R₁ is chosen from linear and branched alkyl and/or alkenyl radicals comprising from 8 to 24 carbon atoms, and alkylphenyl radicals in which the linear or branched alkyl radical comprises from 8 to 24 carbon atoms,

R₂ is chosen from alkylene radicals comprising from 2 to 4 carbon atoms,

G is chosen from sugar units comprising from 5 to 6 carbon atoms,

t is a value ranging from 0 to 10, such as from 0 to 4, and

v is a value ranging from 1 to 15.

By way of non-limiting example, the alkylpolyglycosides according to the present disclosure can be compounds of formula (II) wherein R₁ may be chosen from saturated and unsaturated, linear and branched alkyl radicals comprising from 8 to 18 carbon atoms, t is a value ranging from 0 to 3, such as 0, and G is chosen from glucose, fructose and galactose, for example, glucose. The degree of polymerization, i.e. the value of v in formula (II), can range from 1 to 15, for instance from 1 to 4. The average degree of polymerization can range from 1 to 2, such as from 1.1 to 1.5.

The glycoside linkages between the sugar units are of the 1-6 or 1-4 type. In at least one embodiment, the linkages are of the 1-4 type.

Non-limiting examples of compounds of formula (II) include the products sold by the company Cognis under the names Plantaren® (600 CS/U, 1200 and 2000) or Plantacare® (818, 1200 and 2000). Non-limiting mention may also be made of the products sold by the company SEPPIC under the names TRITON CG 110 (or Oramix® CG 110) and TRITON CG 312 (or Oramix® NS 10), the products sold by the company BASF under the name LUTENSOL GD 70, or else those sold by the company Chem Y under the name AG10 LK. Further non-limiting mention may be made, for example, of the (C₈-C₁₆) alkylpolyglucoside 1,4 in a 53% aqueous solution sold by Cognis under the reference Plantacare® 818 UP.

With respect to the monoglycerol-based or polyglycerol-based surfactants, they can comprise, on average, from 1 to 30 glycerol groups, for instance, from 1 to 10 glycerol groups, such as from 1.5 to 5 glycerol groups.

The monoglycerol-based or polyglycerol-based surfactants can be, by way of non-limiting example, chosen from the compounds of formulae:
RO[CH₂CH(CH₂OH)O]ₘH,
RO[CH₂CH(OH)CH₂O]ₘH, and
RO[CH(CH₂OH)CH₂O]ₘH
wherein R is chosen from saturated and unsaturated, linear and branched hydrocarbon-based radicals comprising from 8 to 40 carbon atoms, for instance from 10 to 30 carbon atoms; m is a number ranging from 1 to 30, such as from 1 to 10, for instance from 1.5 to 6.

R may optionally comprise at least one hetero atom chosen from, for example, oxygen and nitrogen. In addition, R may optionally comprise at least one hydroxyl and/or ether and/or amide group. For example, R may be chosen from C₁₀-C₂₀ alkyl and/or alkenyl radicals that are optionally monohydroxylated or polyhydroxylated.

Use may, for example, be made of the polyglycerol-based (3.5 mol) hydroxylauryl ether sold under the name Chimexane® NF from Chimex.

The at least one nonionic surfactant can be present in the compositions according to the present disclosure in an amount ranging from 5% to 20% by weight, such as from 8% to 18% by weight, relative to the total weight of the composition.

The composition according to the present disclosure may also comprise at least one other surfactant, such as anionic, amphoteric or zwitterionic surfactants, and mixtures thereof.

With respect to the amphoteric or zwitterionic surfactants, non-limiting mention may be made of derivatives of secondary or tertiary aliphatic amines in which the aliphatic radical is chosen from linear and branched chains comprising from 8 to 18 carbon atoms and comprising at least one water-solubilizing anionic group (for example carboxylate, sulphonate, sulphate, phosphate or phosphonate); non-limiting mention may also be made of (C₈-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines.

Among the amine derivatives that may be used as disclosed herein, non-limiting mention may be made of the products sold under the name MIRANOL, as described in U.S. Pat. Nos. 2,528,378 and 2,781,354, and classified in the CTFA dictionary, 3rd edition, 1982, under the names amphocarboxyglycinates and amphocarboxypropionates, having the respective structures:
R₂—CONHCH₂CH₂—N(R₃)(R₄)(CH₂COO⁻)
wherein:

R₂ is chosen from alkyl radicals of R₂—COOH acid present in hydrolysed coconut oil, heptyl radicals, nonyl radicals and undecyl radicals,

R₃ is a beta-hydroxyethyl group and

R₄ is a carboxymethyl group;

and
R′₂—COHNCH₂CH₂—N(B)(C)
wherein:

B is chosen from —CH₂CH₂OX′ radicals,

C is chosen from —(CH₂)_{z}—Y′ radicals, with z=1 or 2,

X′ is chosen from —CH₂CH₂—COOH groups and a hydrogen atom,

Y′ is chosen from —COOH and —CH₂—CHOH—SO₃H radicals, and

R′₂ is chosen from alkyl radicals of an acid R′₂—COOH present in hydrolysed linseed oil or coconut oil, alkyl radicals, such as C₇, C₉, C₁₁ and C₁₃ alkyl radicals, C₁₇ alkyl radicals and their iso form, and unsaturated C₁₇ radicals.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid, disodium cocoamphocarboxyethylhydroxypropyl sulphonate.

By way of example, non-limiting mention may be made of the cocoamphodiacetate sold under the trade name Miranol® C2M concentrate by the company Rhodia Chimie.

The at least one anionic surfactant optionally present may be, for instance, mild anionic surfactants.

Among the mild anionic surfactants that may be used as disclosed herein, non-limiting mention may be made of, for example, the following compounds and salts thereof, and also mixtures thereof:

polyoxyalkylenated alkyl ether carboxylic acids,

polyoxyalkylenated alkylaryl ether carboxylic acids,

polyoxyalkylenated alkylamido ether carboxylic acids, such as those comprising from 2 to 50 ethylene oxide groups,

alkyl-D-galactosiduronic acids,

acylsarcosinates and acylglutamates,

alkylpolyglycoside carboxylic esters.

For example, polyoxyalkylenated alkyl ether carboxylic acids, for instance lauryl ether carboxylic acid (4.5 EO) sold, for example, under the name AKYPO RLM 45 CA from Kao, may be used.

The at least one additional optional surfactant can be present, if they are present, in an amount ranging from 0.1% to 20% by weight, relative to the total weight of the washing composition, such as from 1% to 10% by weight, relative to the total weight of the composition.

The washing composition as disclosed herein may be such that it does not comprise an anionic detergent surfactant of the sulphate type (alkyl sulphate or alkyl ether sulphate, alkylamido ether sulphate). If it does comprise such anionic detergent surfactant, its content is such that the weight ratio of anionic detergent surfactant of alkyl sulphate or alkyl ether sulphate type to the total amount of amphoteric, zwitterionic, mild anionic and nonionic surfactants present, is for example, less than or equal to 1, such as less than or equal to 0.75, and even for instance, less than or equal to 0.5.

The washing composition may also comprise at least one adjuvant conventional in the field, for instance those chosen from the non-exhaustive list such as reducing agents, oxidizing agents, sequestering agents, softeners, antifoams, moisturizers, emollients, basifying agents, plasticizers, sunscreens, direct dyes or oxidation dyes, pigments, mineral fillers, clays, colloidal minerals, nacres, nacrerous agents, fragrances, peptizers, preserving agents, fixing or non-fixing polymers, proteins, vitamins, antidandruff agents, antiseborrhoeic agents, agents for combating hair loss, aliphatic or aromatic alcohols, such as ethanol, benzyl alcohol, modified or unmodified polyols, such as glycerol, glycol, propylene glycol, dipropylene glycol, butylene glycol or butyl diglycol, volatile silicones, mineral, organic or plant oils, oxyethylenated or non-oxyethylenated waxes, paraffins, fatty acids, associative or non-associative thickening polymers, fatty amides, fatty esters, fatty alcohols, etc.

The at least one optional adjuvant can be present, for each adjuvant when present, in an amount ranging from 0.01% to 20% by weight, relative to the weight of the composition.

It should be noted that, if the composition comprises at least one thickener, it can be present in an amount ranging from 0.01% to 20% by weight, relative to the weight of the washing composition, for instance, from 0.01% to 3% by weight, relative to the weight of the washing composition.

The composition according to the present disclosure may also comprise at least one conditioner. When the composition comprises at least one conditioner, it may be chosen from, by way of non-limiting examples, synthetic oils such as poly-α-olefins, fluoro oils, fluoro waxes, fluoro gums, carboxylic acid esters, cationic polymers, silicones, mineral, plant or animal oils, ceramides, and pseudoceramides.

According to one embodiment of the present disclosure, the at least one conditioner may be chosen from cationic polymers and/or volatile or non-volatile silicones. In another embodiment, the at least one conditioner may be chosen from aminosilicones.

When at least one conditioner is present, the at least one conditioner is present in the composition as disclosed herein in an amount ranging from 0.001% to 10% by weight, for instance from 0.005% to 5% by weight, such as from 0.01% to 3% by weight, relative to the total weight of the final composition.

Among the cationic polymers that can be used in the context of the present disclosure, non-limiting mention may be made of quaternary cellulose ether derivatives such as the products sold under the name JR 400 by the company Amerchol, the cationic cyclopolymers, for instance the homopolymers or copolymers of dimethyldiallylammonium chloride, sold under the names Merquat® 100, Merquat® 550 and Merquat® S by the company Nalco, cationic polysaccharides such as the guar gums modified with a 2,3-epoxypropyltrimethylammonium salt, quaternary polymers of vinylpyrrolidone and of vinylimidazole, and mixtures thereof.

The aqueous, for instance physiologically acceptable, medium for the keratin substances can consist of water, or can comprise a mixture of water and at least one cosmetically acceptable organic solvent.

Among the cosmetically acceptable organic solvents that may be used, non-limiting mention may be made, for example, of linear or branched, such as saturated, monoalcohols comprising from 2 to 10 carbon atoms, such as ethyl alcohol or isopropyl alcohol; aromatic alcohols, such as benzyl alcohol or phenylethyl alcohol; polyols or polyol ethers such as, for example, monomethyl, monoethyl and monobutyl ethers of ethylene glycol, propylene glycol or its ethers, such as, for example, propylene glycol monomethyl ether, butylene glycol, dipropylene glycol, hexylene glycol (2-methyl-2,4-pentanediol), neopentyl glycol and 3-methyl-1,5-pentanediol; and also diethylene glycol alkyl ethers, for example C₁-C₄ alkyl ethers, for instance diethylene glycol monoethyl ether or diethylene glycol monobutyl ether, alone or as a mixture.

The at least one solvent can be present in an amount ranging, for example, from 1% to 40% by weight, relative to the total weight of the dye composition, such as from 5% to 30% by weight.

According to the present disclosure, the compositions can be, for example, foaming compositions, i.e. the foaming capacity of the compositions according to the present disclosure, characterized by a foam height, can be greater than or equal to 75 mm; such as greater than or equal to 100 mm, measured according to the modified Ross-Miles method (NF T 73-404/ISO696). The modifications to the method are as follows: the measurement is carried out at a temperature of 22° C. with osmosed water. The concentration of the solution is 2 g/l. The height of the drop is 1 m. The amount of composition that drops is 200 ml. The 200 ml of composition fall in a measuring cylinder having a diameter of 50 mm and containing 50 ml of the test composition. The measurement is carried out 5 minutes after the flow of the composition has stopped.

The composition according to the present disclosure can comprise at least one propellant. The propellant comprises compressed or liquefied gases usually employed for preparing aerosol compositions. Use may be made of, by way of non-limiting example, air, carbon dioxide, compressed nitrogen or else a soluble gas such as dimethyl ether, or hydrocarbons that may be halogenated (for instance, fluorinated) or non-halogenated, and mixtures thereof.

The compositions may be in various galenic forms, such as a lotion, a gel, a spray, an aerosol foam, a pump foam, etc.

According to one embodiment of the present disclosure, the washing composition is packaged in an aerosol device. The device then comprises at least one propellant. The propellant can be chosen from compressed or liquefied gases.

The present disclosure also relates to a process for cosmetically treating keratin substances such as the hair, comprising applying to the hair a composition as defined above, and then in optionally rinsing with water, after an optional leave-in time.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples are intended to illustrate the present disclosure in a non-limiting manner.

### EXAMPLES

### Example 1

The following two shampoo compositions were prepared:

| | | | B | |
|---|---|---|---|---|
| | A | | (comparative) | |
| 53% Cocoglucoside in water | 15 | g AM | 15 | g AM |
| (Plantacare 818 UP-Cognis) | | | | |
| Polyquaternium-10 (JR400-Amerchol) | 0.7 | g AM | 0.7 | g AM |
| Crosslinked copolymer of methacrylic acid | 2.5 | g AM | — | |
| and of ethyl acrylate at 30% in water | | | | |
| (Carbopol AquaSF-1 from Noveon) | | | | |
| Crosslinked polyacrylic acid | — | | 2.5 | g AM |
| (Carbopol 980 from Noveon) | | | | |
| HCl qs | pH 6 | | pH 6 | |
| Water qs | 100 | g | 100 | g |

The quality of the foam of the formulae was evaluated on locks of hair (rate of initiation, not too aerated in nature, abundance and hold).

The application conditions were as follows:

After having been wetted with water (3 passages between the fingers under water), each lock was dried between two fingers and an amount of 0.4 g of shampoo/gram of hair was applied along the lock of hair (from the root to the tip evenly).

Foaming was then produced by gently massaging the lock between two fingers along its length for 15 seconds from top to bottom (without making knots). The rate of initiation of the shampoo was then observed.

The lock was then rolled around the fingers and placed in a plastic cupule.

The abundance of foam and the aerated nature of each of the shampoos prepared was then observed.

The shampoo was then left in for a period timed at 5 min for each application, and the hold of the foam was observed.

| | A | B |
|---|---|---|
| Visual appearance at 24 h | clear fluid gel | granular opaque gel |
| Stability 1 week at 45° C. | stable | unstable (hardens) |

The composition comprising the Carbopol Aqua SF1 (composition A) obtained altogether:

an aesthetic appearance: clear fluid gel,

good stability and having a viscosity sufficient to allow comfortable application to the hair,

excellent foam qualities (rate of initiation of the foam, aerated nature of the foam, abundance and hold of the foam)

### Example 2

The following compositions were prepared:

| | | | D | |
|---|---|---|---|---|
| | C | | (comparative) | |
| 53% Cocoglucoside in water | 15 | g AM | — | |
| (Plantacare 818 UP-Cognis) | | | | |
| 25% Sodium laureth sulphate | — | | 15 | g AM |
| (Texapon N 702) | | | | |
| Polyquaternium-10 | 0.7 | g AM | 0.7 | g AM |
| (JR400-Amerchol) | | | | |
| Crosslinked copolymer of methacrylic | 1.8 | g AM | 1.8 | g AM |
| acid/ethyl acrylate at 30% in water | | | | |
| (Carbopol AquaSF-1 from Noveon) | | | | |
| HCl qs | pH 6 | | pH 6 | |
| Water | qs 100 | g | qs 100 | g |

| | | |
|---|---|---|
| Appearance at 24 h | transparent | opalescent |
| | homogeneous | precipitate |
| | fluid gel | |

Shampoo C comprising a large amount of nonionic surfactant obtained a shampoo that had an aesthetic (transparent) visual appearance and was stable, whereas shampoo D with a high content of anionic surfactant was neither transparent nor stable.

Furthermore, shampoo C exhibited sufficient and stable thickening, and good foam, cosmetic and detergent properties.

It is also noted, on hair dyed with Majicontrast Rouge, that the bleeding of the color in the water and in the foam was less substantial in the case of shampoo of the present disclosure. The coloration had improved hold.

### Example 3

The following compositions were prepared:

| | Shampoo E | | Shampoo F | |
|---|---|---|---|---|
| 53% Cocoglucoside in water | 15 | g AM | — | |
| (Plantacare 818 UP-Cognis) | | | | |
| Polyglyceryl-3 hydroxylauryl ether, | — | | 15 | g AM |
| Chimexane NF from Chimex | | | | |
| Polyquaternium-10 (JR400-Amerchol) | 0.7 | g AM | 0.7 | g AM |
| Crosslinked copolymer of methacrylic | 1.8 | g AM | 1.8 | g AM |
| acid/ethyl acrylate at 30% in water | | | | |
| (Carbopol AquaSF-1 from Noveon) | | | | |
| HCl qs | pH 6 | | pH 6 | |
| Water | qs 100 | g | qs 100 | g |

| | | |
|---|---|---|
| Appearance at 24 h | transparent | transparent |
| | fluid gel | fluid gel |

The compositions were all satisfactory from both the point of view of the appearance, and from the point of view of the application to the hair and of the cosmetic results.

### Example 4

| | Shampoo G | |
|---|---|---|
| 53% (C₈-C₁₆) Cocoglucoside in water | 15 | g AM |
| (Plantacare 818 UP-Cognis) | | |
| Polyquaternium-10 (JR400-Amerchol) | 0.7 | g AM |
| Crosslinked copolymer methacrylic acid | 2 | g AM |
| and of ethyl acrylate at 28% in water | | |
| (Aculyn 33 from Rohm & Haas) | | |
| Amino-2-methyl-2-propanol qs | pH 7 | |
| Water qs | 100 | g |

### Example 5

The following two shampoo compositions were prepared:

| | | | I | |
|---|---|---|---|---|
| | H | | (comparative) | |
| 53% Cocoglucoside in water | 15 | g AM | 15 | g AM |
| (Plantacare 818 UP-Cognis) | | | | |
| Polyquaternium-10 (JR400-Amerchol) | 0.7 | g AM | 0.7 | g AM |
| Crosslinked copolymer of methacrylic | 2.5 | g AM | — | |
| acid and of ethyl acrylate at 30% | | | | |
| in water (Carbopol AquaSF-1 from | | | | |
| Noveon) | | | | |
| Crosslinked copolymer of methyl | — | | 2.5 | g AM |
| vinyl ether/maleic anhydride | | | | |
| (Styleze QM from ISP) | | | | |
| HCl qs | pH 6 | | pH 6 | |
| Water qs | 100 | g | 100 | g |

The quality of the foam of the formulae was evaluated on locks of hair (rate of initiation, not too aerated in nature, abundance and hold).

The application conditions were as follows:

After having been wetted with water (3 passages between the fingers under water), each lock was dried between two fingers and an amount of 0.4 g of shampoo/gram of hair was applied along the lock of hair (from the root to the tip evenly).

Foaming was then produced by gently massaging the lock between two fingers along its length for 15 seconds from top to bottom (without making knots). The rate of initiation of the shampoo was then observed.

The lock was then rolled around the fingers and placed in a plastic cupule.

The abundance of foam and the aerated nature of each of the shampoos prepared was then observed.

The shampoo was then left in for a period timed at 5 min for each application, and the hold of the foam was observed.

| | H | I |
|---|---|---|
| Visual appearance at 24 h | clear fluid gel | White granular thick |
| | | no homogeneous gel |

The composition comprising the Carbopol Aqua SF1 (composition H) obtained altogether:

an aesthetic appearance: clear fluid gel,

good stability and having a viscosity sufficient to allow comfortable application to the hair,

excellent foam qualities (rate of initiation of the foam, aerated nature of the foam, abundance and hold of the foam)

The composition I was a thick gel difficult to spread over hair. Foam initiated slowly and it was less abundant.

1. A detergent cosmetic composition comprising, in a physiologically acceptable aqueous medium,
at least one nonionic surfactant, present in an amount ranging from 5% to 20% by weight, relative to the total weight of the composition, chosen from:
glycerol-based fatty alcohols;
glycerol-based fatty amides;
oxyalkylenated plant oils;
alkylpolyglycosides; and
derivatives of N-alkylglucamine; and
at least one crosslinked anionic copolymer, present in an amount ranging from 0.01% to 10% by weight, relative to the total weight of the composition, of vinyl carboxylic acid (monomer A) and of a monomer having at least one α,β-ethylenic unsaturation (monomer B) chosen from the monomers of formulae:
CH₂═CXY i)
wherein X is a hydrogen and Y is chosen from —COOR, —C₆H₄R′, —CN, —CONH₂, —NHCOCH₃, —CONHC(CH₃)₃, and —CON(CH₃)₂ groups, or
X is a CH₃ group and Y is chosen from —COOR, —C₆H₄R′, —CN and —CH═CH₂ groups,
R is chosen from C₁-C₈ alkyl and C₂-C₈ hydroxyalkyl groups,
R′ is chosen from a hydrogen atom, and C₁-C₈ alkyl and C₂-C₈ hydroxyalkyl groups;
CH₂═CH(OCOR¹) ii)
wherein R¹ is chosen from C₁-C₈ alkyl groups; and
CH₂═CH₂ or CH₂═CHCH₃. iii)

2. The detergent cosmetic composition according to claim 1, wherein the monomer B is chosen from the monomers of formula i).

3. The detergent cosmetic composition according to claim 2, wherein the monomer B is chosen from the monomers of formula i) wherein R is chosen from C₁-C₄ alkyl and C₂-C₄ hydroxyalkyl groups.

4. The detergent cosmetic composition according to claim 3, wherein the monomer B is chosen from methyl acrylate, ethyl acrylate, n-butyl acrylate, styrene, acrylamide, N,N-dimethylacrylamide, tert-butylacrylamide, methyl methacrylate, ethyl methacrylate, and n-butyl methacrylate.

5. The detergent cosmetic composition according to claim 4, wherein the monomer B is ethyl acrylate.

6. The detergent cosmetic composition according to claim 1, wherein the monomer A is chosen from acrylic acid, methacrylic acid, itaconic acid, fumaric acid, crotonic acid, maleic acid and mixtures thereof.

7. The detergent cosmetic composition according to claim 6, wherein the monomer A is chosen from methacrylic acid and acrylic acid.

8. The detergent cosmetic composition according to claim 1, wherein, in the copolymer, the monomer A is present in an amount ranging from 20% to 80% by weight, relative to the total weight of the copolymer.

9. The detergent cosmetic composition according to claim 8, wherein, in the copolymer, the monomer A is present in an amount ranging from 35% to 60% by weight, relative to the total weight of the copolymer.

10. The detergent cosmetic composition according to claim 1, wherein, in the copolymer, the monomer B is present in an amount ranging from 15% to 80% by weight, relative to the total weight of the copolymer.

11. The detergent cosmetic composition according to claim 10, wherein, in the copolymer, the monomer B is present in an amount ranging from 40% to 65% by weight, relative to the total weight of the copolymer.

12. The detergent cosmetic composition according to claim 1, wherein the at least one crosslinked copolymer is crosslinked with at least one ethylenically polyunsaturated crosslinking agent.

13. The detergent cosmetic composition according to claim 12, wherein the at least one crosslinking agent is present in an amount ranging from 0.01% to 5% by weight, relative to the total weight of the copolymer.

14. The detergent cosmetic composition according to claim 13, wherein the at least one crosslinking agent is present in an amount ranging from 0.05% to 1% by weight, relative to the total weight of the copolymer.

15. The detergent cosmetic composition according to claim 1, wherein the at least one crosslinked copolymer is a crosslinked copolymer of methacrylic acid and of C₁-C₄ alkyl acrylate.

16. The detergent cosmetic composition according to claim 15, wherein the at least one crosslinked copolymer is a crosslinked copolymer of methacrylic acid and of ethyl acrylate.

17. The detergent cosmetic composition according to claim 1, wherein the at least one crosslinked copolymer is present in an amount ranging from 0.01% to 10% by weight, relative to the total weight of the composition.

18. The detergent cosmetic composition according to claim 17, wherein the at least one crosslinked copolymer is present in an amount ranging from 0.1% to 5% by weight, relative to the total weight of the composition.

19. The detergent cosmetic composition according to claim 1, wherein the at least one nonionic surfactant is chosen from:
glycerol-based fatty alcohols; and
alkylpolyglycosides.

20. The detergent cosmetic composition according to claim 19, wherein the at least one nonionic surfactant is chosen from alkylpolyglycosides, monoglycerol-based surfactants and polyglycerol-based surfactants.

21. The detergent cosmetic composition according to claims 19, wherein the at least one nonionic surfactant is a alkylpolyglycoside chosen from those of formula:
R₁O—(R₂O)ₜ(G)ᵥ
wherein
R₁ is chosen from linear and branched alkyl and/or alkenyl radicals comprising from 8 to 24 carbon atoms, or an alkylphenyl radical in which the linear or branched alkyl radical comprises from 8 to 24 carbon atoms,
R₂ is chosen from alkylene radicals comprising from 2 to 4 carbon atoms,
G is a sugar unit comprising from 5 to 6 carbon atoms,
t is a value ranging from 0 to 10, and
v is a value ranging from 1 to 15.

22. The detergent cosmetic composition according to claim 21, wherein t is a value ranging from 0 to 4.

23. The detergent cosmetic composition according to claim 20, wherein the monoglycerol-based or polyglycerol-based surfactants are chosen from the compounds of formulae:
RO[CH₂CH(CH₂OH)O]ₘH,
RO[CH₂CH(OH)CH₂O]ₘH, and
RO[CH(CH₂OH)CH₂O]ₘH
wherein R is chosen from saturated and unsaturated, linear and branched hydrocarbon-based radicals comprising from 8 to 40 carbon atoms; and m is a number ranging from 1 to 30; R may optionally comprise at least one entity chosen from hetero atoms and/or hydroxyl and/or ether and/or amide groups.

24. The detergent cosmetic composition according to claim 23, wherein R is chosen from C₁₀-C₂₀ alkyl and/or alkenyl radicals that are optionally monohydroxylated or polyhydroxylated.

25. The detergent cosmetic composition according to claim 23, wherein R comprises at least one hetero atom.

26. The detergent cosmetic composition according to claim 1, wherein the at least one nonionic surfactant is present in the composition in an amount ranging from 5% to 20% by weight, relative to the total weight of the composition.

27. The detergent cosmetic composition according to claim 26, wherein the at least one nonionic surfactant is present in the composition in an amount ranging from 8% to 18% by weight, relative to the total weight of the composition.

28. The detergent cosmetic composition according to claim 1, further comprising at least one additional surfactant.

29. The detergent cosmetic composition according to claim 28, wherein the at least one additional surfactant is present in an amount ranging from 0.1% to 20% by weight, relative to the total weight of the composition.

30. The detergent cosmetic composition according to claim 29, wherein the at least one additional surfactant is present in an amount ranging from 1% to 10% by weight, relative to the total weight of the composition.

31. The detergent cosmetic composition according to claim 1, further comprising at least one conditioner.

32. The detergent cosmetic composition according to claim 31, wherein the at least one conditioner is chosen from poly-α-olefins, fluoro oils, fluoro waxes, fluoro gums, carboxylic acid esters, silicones, cationic polymers, mineral oils, plant oils, animal oils, ceramides, and pseudoceramides.

33. The detergent cosmetic composition according to claim 32, wherein the at least one conditioner is chosen from cationic polymers and silicones.

34. The detergent cosmetic composition according to claim 33, wherein the at least one conditioner is an aminosilicone.

35. The detergent cosmetic composition according to claim 31, wherein the at least one conditioner is present in an amount ranging from 0.001% to 10% by weight, relative to the total weight of the composition.

36. The detergent cosmetic composition according to claim 35, wherein the at least one conditioner is present in an amount ranging from 0.01% to 3% by weight, relative to the total weight of the composition.

37. The detergent cosmetic composition according to claim 1, wherein the composition is packaged in an aerosol device.

38. A method for limiting the bleeding of color and/or improving the protection of the coloration of keratin fibers while washing them comprising
applying to the fibers a composition comprising, in a physiologically acceptable aqueous medium:
at least one nonionic surfactant, present in an amount ranging from 5% to 20% by weight, relative to the total weight of the composition, chosen from:
glycerol-based fatty alcohols;
glycerol-based fatty amides;
oxyalkylenated plant oils;
alkylpolyglycosides; and
derivatives of N-alkylglucamine; and
at one crosslinked anionic copolymer, present in an amount ranging from 0.01% to 10% by weight, relative to the total weight of the composition, of vinyl carboxylic acid (monomer A) and of a monomer having at least one α,β-ethylenic unsaturation (monomer B) chosen from the monomers of formulae:
CH₂═CXY i)
wherein X is a hydrogen and Y is chosen from —COOR, —C₆H₄R′, —CN, —CONH₂, —NHCOCH₃, —CONHC(CH₃)₃, and —CON(CH₃)₂ groups, or
X is a CH₃ group and Y is chosen from —COOR, —C₆H₄R′, —CN and —CH═CH₂ groups,
R is chosen from C₁-C₈ alkyl and C₂-C₈ hydroxyalkyl groups,
R′ is chosen from a hydrogen atom, and C₁-C₈ alkyl and C₂-C₈ hydroxyalkyl groups;
CH₂═CH(OCOR¹) ii)
wherein R′ is chosen from C₁-C₈ alkyl groups; and
CH₂═CH₂ or CH₂═CHCH₃, iii)
wherein the composition is applied to the fibers in an effective amount sufficient to limit the bleeding of color and/or improve the protection of the coloration of keratin fibers.

39. A process for cosmetically treating keratin substances comprising
applying, to the keratin substances, a composition comprising, in a physiologically acceptable aqueous medium:
at least one nonionic surfactant, present in an amount ranging from 5% to 20% by weight, relative to the total weight of the composition, chosen from:
glycerol-based fatty alcohols;
glycerol-based fatty amides;
oxyalkylenated plant oils;
alkylpolyglycosides; and
derivatives of N-alkylglucamine; and
at one crosslinked anionic copolymer, present in an amount ranging from 0.01% to 10% by weight, relative to the total weight of the composition, of vinyl carboxylic acid (monomer A) and of a monomer having at least one α,β-ethylenic unsaturation (monomer B) chosen from the monomers of formulae:
CH₂═CXY i)
wherein X is a hydrogen and Y is chosen from —COOR, —C₆H₄R′, —CN, —CONH₂, —NHCOCH₃, —CONHC(CH₃)₃, and —CON(CH₃)₂ groups, or
X is a CH₃ group and Y is chosen from —COOR, —C₆H₄R′, —CN and —CH═CH₂ groups,
R is chosen from C₁-C₈ alkyl and C₂-C₈ hydroxyalkyl groups,
R′ is chosen from a hydrogen atom, and C₁-C₈ alkyl and C₂-C₈ hydroxyalkyl groups;
CH₂═CH(OCOR¹) ii)
wherein R¹ is chosen from C₁-C₈ alkyl groups; and
CH₂═CH₂ or CH₂═CHCH₃, iii)
and then optionally rinsing the keratin substances with water.
